(19) United States
(12) Patent Application Publication
(10) Pub. No.: US 2018/0266957 A1
(43) Pub. Date: Sep. 20, 2018

(54) SYSTEM AND METHOD FOR CORRECTING FOR PHOSPHORESCENCE CONTRIBUTION IN LASER SCANNING IMAGING

(71) Applicant: THE BOARD OF REGENTS FOR OKLAHOMA STATE UNIVERSITY, STILLWATER, OK (US)

(72) Inventor: Eduardo Gardenali Yukihara, Stillwater, OK (US)

(21) Appl. No.: 15/542,519

(22) PCT Filed: Jan. 15, 2016
(86) PCT No.: PCT/US16/13653

§ 371 Date: Jul. 10, 2017

Related U.S. Application Data
(60) Provisional application No. 62/103,605, filed on Jan. 15, 2015.

(51) Int. Cl. G01N 21/64; G06K 9/40; A61B 1/04; G02B 21/00

(52) U.S. Cl. CPC G01N 21/6456; G06K 9/40; G02B 21/002; A61B 1/043; G01N 21/6428

(57) ABSTRACT

There is provided a method for correcting “pixel bleeding” caused by slow luminescence centers in laser scanning imaging (e.g., X-ray imaging using photostimulable phosphors and 2D dosimetry using optically stimulated luminescence). An embodiment uses a deconvolution procedure that takes into account the lifetime of the slow luminescence center and can be further constrained by the detection of fast and slow luminescence centers and combining line scans in opposite directions. An approach has been tested using simulated data and demonstrated experimentally by applying it to image reconstruction of two types of Al₂O₃ X-ray detector films (Al₂O₃:C and Al₂O₃:C,Mg), whose use in 2D dosimetry in conjunction with laser-scanning readout has so far been prevented by slow luminescence centers (35 ms lifetime from F-centers). We show that the algorithm allows the imaging using Al₂O₃ detectors 300 times faster than generally allowed considering the lifetime of the main luminescence centers.

### CROSS REFERENCE TO RELATED APPLICATIONS

This application claims the benefit of U.S. Provisional Patent Application Ser. No. 62/103,605 filed on Jan. 15, 2015, and incorporates said provisional application by reference into this document as if fully set out at this point.

### TECHNICAL FIELD

This disclosure relates generally to laser scanning imaging operations and, more particularly, to system and methods for correcting for phosphorescence contribution in laser scanning imaging.

### BACKGROUND

Active or flying-spot laser scanning combined with luminescence detection is used in a variety of imaging application, including scanning confocal microscopy (Sheppard and Shotton, 1997), computed radiography (Rowlands, 2002; von Seggern, 1999), particle track detection (Akselrod et al., 2006), and two dimensional ionizing radiation dose mapping (Ahmed et al., 2014). Active laser scanning refers to the illumination of the detector with a “flying laser spot”, created by the deflection of the laser beam by, for example, rotating mirrors, rotating mirror polygons, and galvanometers (Beiser and Johnson, 2010).

In computed radiography and two-dimensional dose mapping, the laser spot stimulates the recombination of a population of trapped charges created by ionizing radiation, which results in phosphorescence called optically stimulated luminescence (OSL) or photostimulated luminescence (PSL) (Rowlands, 2002; von Seggern, 1999). The luminescence is detected using a light transducer, typically a photomultiplier tube, and the image is obtained by associating the luminescence intensity with the position of the laser spot. For purposes of the instant disclosure, the term ‘OSL” should be broadly construed to include both OSL and PSL.

In these applications, the signal must originate from luminescence centers having short luminescence lifetime, i.e., luminescence which decays faster than the typical pixel dwell time (time the laser spot spend on any given position). If the luminescence lifetime is longer than the pixel dwell time, the molecules or defects excited when the laser is stimulating a specific pixel will continue to emit after the laser has moved to other positions in the sample or detector. Since the light transducers are not typically position sensitive (to discriminate the light coming from different pixels), the result is that luminescence will bleed into neighboring pixels, degrading the image resolution or limiting the scan rate and readout time (von Seggern, 1999). This type of bleeding, which here we call pixel bleeding, should not be confused with spectral bleeding, spectral bleed-through, or cross-over, which refers to the phenomenon in which the signal from a luminophore is detected in the detector channel of a different luminophore because of overlap of the fluorescence spectra (Claxton et al., 2006).

Pixel bleeding has been a major obstacle in the development of 2D dosimetry systems using OSL. On the one hand, OSL from carbon-doped aluminum oxide (Al₂O₃:C) has been widely used in personal and medical dosimetry (Bøtter-Jensen et al., 2003; McKeever, 2001; Yukihara and McKeever, 2008) because of its high sensitivity to ionizing radiation, relatively low effective atomic number (Z_{eff}˜11.3), signal stability, independence on dose rate, and minimal energy dependence in megavoltage photon and electron beams. On the other hand, the long luminescence lifetime (˜35 ms) of the main luminescence centers in Al₂O₃:C, F-centers (Akselrod et al., 1998; Lee and Crawford Jr., 1979), has prevented the use of Al₂O₃:C for 2D dosimetry using active laser-scanning. If one uses the criterion of stimulating each pixel for three time constants (to avoid image deterioration due to pixel bleeding) (von Seggern, 1999), or 100 ms in this case, the readout of a 15 cm×15 cm detector with a 0.1 mm scan resolution (1500×1500 pixels) would take 65 hours. Other imaging approaches, such as uniform illumination of the detector and OSL imaging using CCD cameras, are still possible, but laser-scanning offers advantages such as lower cost of the basic components, higher versatility given by the selection of scan rate and scan area, and higher sensitivity provided by the use of photomultiplier tubes as the light transducer.

Other materials have also been investigated for 2D dosimetry using OSL, including storage phosphors used in computed radiography (Olch, 2005), BeO (Jahn et al., 2010; Jahn et al., 2011), SrS (Idri et al., 2004), and KCl (Han et al., 2009; Li et al., 2013). Several of these materials have short luminescence lifetime, but the main shortcoming is the high effective atomic number Z_{eff}. In the case of storage phosphors, the luminescence lifetime is <1 μs, but Z_{eff}>30-50 (e.g., BaFCl:Eu²⁺, BaFBr:Eu²⁺, RbBr:Tl²⁺, etc.) (Rowlands, 2002). For SrS the effective atomic number is Z_{eff}=34.6 (Yukihara and McKeever, 2011), and for KCl it is Z_{eff}=18 (Han et al., 2009). These relatively high effective atomic numbers introduce a photon energy dependence, particularly in the presence of scattered, low energy photons (Han et al., 2009; Olch, 2005). BeO is the most tissue-equivalent OSL materials (Z_{eff}=7.2), but the material has only be used in rigid plates, probably because of the toxicity of BeO powder (Jahn et al., 2010; Jahn et al., 2011).

Given the obstacles outlined above, it would be advantageous to develop a 2D dosimetry system based on the OSL from Al₂O₃:C. However, a technique is needed to correct for pixel bleeding of the slow F-center luminescence.

Before proceeding to a description of the present invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY OF THE INVENTION

There is taught herein a method to correct for “pixel bleeding” caused by slow luminescence centers in laser scanning imaging (e.g., X-ray imaging using photostimulable phosphors and 2D dosimetry using optically stimulated luminescence). An embodiment of the technique taught herein is based on a deconvolution procedure that takes into account the lifetime of the slow luminescence center and can be further constrained by combining the detection of fast and slow luminescence centers and combining the line scan in opposite directions. The algorithm has been tested using simulated data and demonstrated experimentally by applying it to image reconstruction of two types of Al₂O₃ X-ray detector films (Al₂O₃:C and Al₂O₃:C,Mg). Al₂O₃:C,Mg is a modified form of Al₂O₃:C containing a higher proportion of F⁺-center emission relative to F-center emission than Al₂O₃:C (Rodriguez et al., 2011).

The method could be of interest to other laser scanning applications in which it is desirable to correct for or use the information provided by luminescence centers characterized by luminescence lifetime longer than the pixel dwell time. Examples include fluorophores having microsecond fluorescence lifetimes, such as lanthanide chelates. These are of interest for confocal microscopy because of the potential to use time-resolved detection to improve the signal-to-noise ratio and to discriminate autofluorescence from the biological samples, which have short luminescence lifetime (Tsien et al., 2006).

According to an embodiment, there is provided a method of laser scanning imaging a sample containing a luminescent material (box 1105), comprising the steps of: selecting or identifying a response function for said luminescent material (box 1110); performing a laser scan of said sample (box 1115), thereby obtaining a plurality of luminescence values; using said response function of said luminescent material to form a design matrix corresponding to said laser scan (box 1120); and, using said design matrix and said plurality of luminescence values to calculate an adjusted profile for said sample (box 1125).

According to another embodiment, there is provided a method of performing 2D dosimetry on a sample of a luminescent material, comprising the steps of: selecting a response function for said luminescent material; obtaining a plurality of laser scans of the sample, thereby obtaining a plurality of luminescence values; using said response function of said luminescent material to form a design matrix corresponding to each of said plurality of laser scans; and, using said design matrix and said plurality of luminescence values to calculate an adjusted OSL dose profile for said sample using deconvolution.

According to still another embodiment, there is provided a method of laser scanning imaging a sample containing a luminescent material, comprising the steps of: selecting a response function for said luminescent material; performing a first laser scan of said sample in a first direction, thereby obtaining a first plurality of luminescence values; performing a second laser scan of said sample in a direction opposite said first direction, thereby obtaining a second plurality of luminescence values; using said response function of said luminescent material to form a design matrix corresponding to said first laser scan direction and said second laser scan direction; and, using said design matrix and said first and second plurality of luminescence values to calculate an adjusted profile for said sample.

In some embodiments the first direction will be a positive direction and the second scan direction will be in a negative direction.

The foregoing has outlined in broad terms some of the more important features of the invention disclosed herein so that the detailed description that follows may be more clearly understood, and so that the contribution of the instant inventors to the art may be better appreciated. The instant invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### BRIEF DESCRIPTION OF THE DRAWINGS

These and further aspects of the invention are described in detail in the following examples and accompanying drawings.

FIG. 1 contains a schematic illustration of a signal S(t) for an embodiment as it appears when the laser scans a one-dimensional detector with dwell time Δt, characterized by dose zero everywhere except between pixels 300-500, where the dose is non-zero and constant. The lifetime of the luminescence centers was assumed to be 30 Δt.

FIG. 2 contains a diagram of possible physical process involved in luminescence imaging for an embodiment: (a) photostimulated luminescence in which a fluorophor (luminescence molecule or luminescence center in a crystalline matrix) is stimulated from the ground to an excited state with a probability p, and relaxes from another excited state to the ground state with a probability τ⁻¹, where τ is the lifetime of the luminescence center (assuming no non-radiative relaxation); (b) optically stimulated process in which trapped charges (concentration n) are stimulated to the conduction band with probability p, recombining with their counterparts creating a defect in the excited state, which relaxes to the ground state with probability τ⁻¹, where τ is again the lifetime of the luminescence center (assuming no non-radiative relaxation).

FIG. 3 contains an illustration of a theoretical luminescence signal for an embodiment resulting from excitation/stimulation of luminescence centers for a period of 1 μs, for luminescence centers characterized by lifetimes of 10 ns, 1 μs, or 3 μs. The luminescence decay tail after the excitation/stimulation is responsible for the pixel bleeding discussed in this work.

FIG. 4 contains an example of a design matrix column X_{i,100}, which gives the signal for a unit dose at j=100 in one scan direction (from i=1 to 1000 and then in the other scan direction from i=1001 to 2000). In these calculations it is assumed that there is a pause between the two scans, so that luminescence in one scan will not bleed into the next one. R=0, α=10², τ=10 Δt.

FIG. 5 contains an illustration of a simulated signal, dose profiles (initial and recovered) and residual considering signal of 10⁶ counts/pixel in the non-zero dose region, and different luminescence lifetimes for an embodiment: (a) τ=1 Δt (simulation set A-1, see Table 1), (a) τ=10 Δt (simulation set A-2, see Table 1), (a) τ=100 Δt (simulation set A-3, see Table 1).

FIG. 6 contains an illustration of PMT signal, dose obtained with the algorithm, and residual (difference between PMT signal and fitted signal) according to an embodiment for: (a) Al₂O₃:C measured using the F-center emission (Hoya B-370+Schott BG-39 filter); (b) Al₂O₃:C measured using a combination of F- and F⁺-center emission (Hoya U-340 filter); and (c) Al₂O₃:C,Mg measured using a combination of F- and F⁺-center emission (Hoya U-340 filter). The PMT signals correspond to a pair of rows passing through the middle of the OSL detectors from FIG. 10 (discussed in detail below). The value of R used in the algorithm is the one that minimizes the residuals: R=0.02 for (a), R=0.4 for (b), and R=0.815 for (c). For Al₂O₃:C τ=35.9 ms and Al₂O₃:C τ=33.0 ms were used.

FIG. 7 illustrates an embodiment of a simulated signal in two scan directions, dose profiles (ideal and recovered) and residual considering signal of 10³ in the non-zero dose region, luminescence lifetimes τ=10 Δt and (a) only slow luminescence center (simulation set B-2, see Table 1) or (b) a combination of fast and slow luminescence centers (R=0.3) (simulation set C-2, see Table 1).

FIG. 8 contains an example comparison between the recovered dose response in two simulations considering signal of 10² in the non-zero dose region, luminescence lifetimes τ=100 Δt and a combination of slow and fast luminescence center (R=0.3) (simulation set E-3, see Table 1): (a) obtained without weighting (σ²=1); and (b) obtained using the (reciprocal of) uncertainties as weight (σ²=y).

FIG. 9 contains an illustration of an example of a simulated signal in (a) one (simulation set SC-2, see Table 2) or (b) two scan directions (simulation set C-3, see Table 1), dose profiles (ideal and recovered) and residual considering signal of 10³ in the non-zero dose region, luminescence lifetimes τ=100 Δt and a combination of fast and slow luminescence centers (R=0.3).

FIG. 10 contains examples of images obtained using three OSL discs (7 mm in diameter) irradiated with ˜1 Gy: (a) Al₂O₃:C measured using the F-center emission (Hoya B-370+Schott BG-39 filter) before correction and (b) after correction for pixel bleeding; (c) Al₂O₃:C measured using a combination of F- and F⁺-center emission (Hoya U-340 filter) before correction and (d) after correction for pixel bleeding; (e) Al₂O₃:C,Mg measured using a combination of F- and F⁺-center emission (Hoya U-340 filter) before correction and (f) after correction for pixel bleeding. The images were obtained scanning the laser in alternating directions; each row corresponds to 1024 points and the image is a composite of 251 rows separated by 0.167 mm. The dwell time in each pixel is 327 μs, resulting in a total scan time of 1.5 min. The value of R used in the algorithm is the one that minimizes the residuals: R=0.022 for (a) and (b), R=0.4 for (c) and (d), and R=0.815 for (e) and (f). For Al₂O₃:C we used τ=35.9 ms and for Al₂O₃:C we used τ=33.0 ms.

FIG. 11 contains an operating logic suitable for use with an embodiment.

### DETAILED DESCRIPTION

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

### Fluorescence Laser Scanning

Assume a one-dimensional luminescence detector (or a single row in a two-dimensional detector) irradiated with a given dose profile. The signal S(t) detected when a laser scans the detector, exciting luminescence center which decays exponentially with a lifetime τ, is given by the convolution of the dose profile with the exponential decay of the luminescence:

$$S ( t ) = {\int }_{0}^{t} f ( t - s ) g ( s ) ds \qquad ( 1 )$$

where f(t) is the dose profile at an instant t, when the laser is at position x(t), and g(t) is the (normalized) exponential decay with lifetime τ, given by:

g(t)∝e^{-1/τ}. (2)

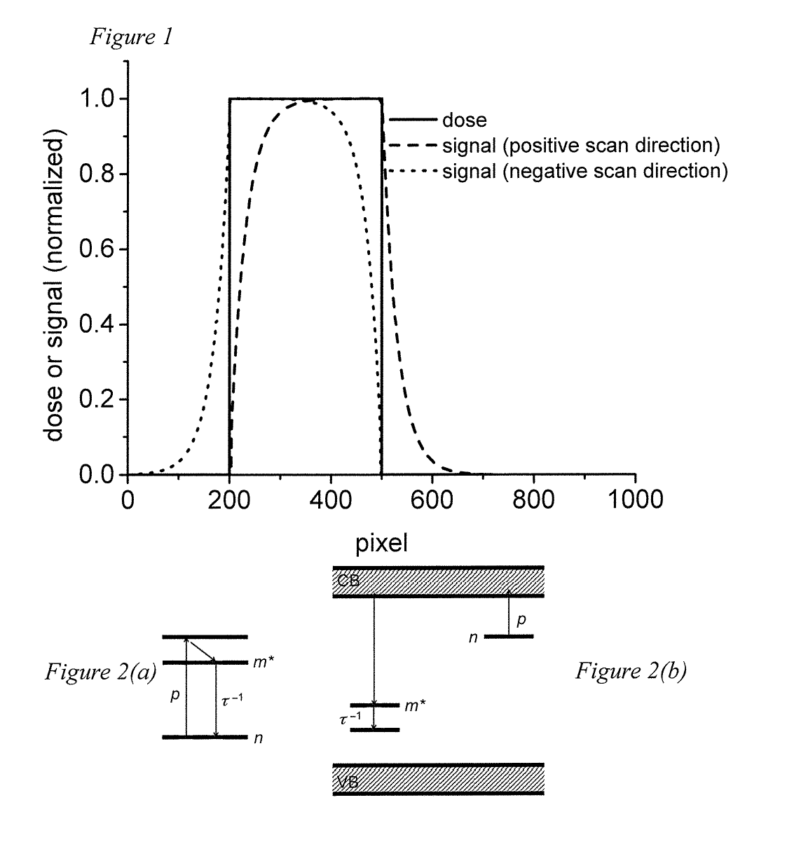

Consider for example a detector with 1000 pixels characterized by a constant dose from pixel 200 to 500 and zero otherwise, scanned by a laser with a dwell time Δt, and having luminescence centers characterized by a lifetime 30 times the dwell time (30 Δt). FIG. 1 shows the signal S(t) when the laser scans the detector in the positive scan direction, i.e., from pixel 1 towards pixel 1000. The signal is zero up to the instant when the laser starts scanning the region of non-zero dose at t=200 Δt, at which point the signal starts to increase: in addition to the signal excited in each new pixel, the pixels previously excited are still contributing to the signal. In this case, this contribution builds up until an equilibrium level, when the additional luminescence from new scanned pixels compensates the decay of the luminescence from previously scanned pixels. As the laser leaves the region of non-zero dose at t=500 Δt, the signal decays with a lifetime τ, because there are no new contributions to the luminescence.

This figure also shows the signal S(t) according to the current example when the detector is scanned in the opposite (negative) scan direction (from pixel 1000 towards pixel 1). The signal shows the same profile, except that in this case the laser enters the non-zero dose region at t=500 Δt (pixel 500) and leaves it at t=800 Δt (pixel 200).

If the signal S is now plotted as a function of laser spot position according to an embodiment, the profiles shown in FIG. 1 are obtained. In this figure, both scan profiles are compared to the original dose in the detector, making evident the distortion introduced by the long luminescence lifetime of the luminescence centers.

In practice, one wants to obtain the dose profile as a function of position f(x) using the signal S(t) to find f(t) by deconvolution, and combining the solution f(t) with the laser position x(t). This deconvolution problem is ill-posed and typically does not have a unique solution even in the absence of noise. The approach proposed according to an embodiment combines knowledge of the luminescence lifetime τ, which determines g(t); and the least squares method. Furthermore, and continuing with an embodiment, results from this approach can be improved by: (a) detection of a combination of slow luminescence centers (F-center) and fast luminescence centers (F⁺-centers); and, (b) by combining the scan of the detector in two opposite directions. Furthermore, this approach can also be used to account for more than one phosphorescence component, introduced for example by other trapping centers (shallow traps) that temporarily capture the stimulated charges before they can recombine.

The effectiveness of an embodiment of the teachings herein in the presence of noise are illustrated using simulations. Additionally, a variation of the correction algorithm is tested using experimental data on Al₂O₃:C and Al₂O₃:C,Mg.

### Lifetime of the Luminescence Process

Al₂O₃:C is an interesting system because its OSL signal has two components: the main one that emits at ˜420 nm characterized by a 35 ms lifetime associated with F-centers and a second one emitting at ˜335 nm characterized by short luminescence lifetime (<7 ns) likely associated with F⁺-center.

In general, equations for the luminescence process can be obtained by considering a luminescence center that can be excited with a probability p, which depends on the transition probability and laser intensity, and decays from the excited state to the ground state with a probability τ⁻¹, where τ is the lifetime of the excited state. The excited state can be populated due to excitation of fluorescent molecules from the ground state in case of fluorescence (FIG. 2(a)), or due to recombination of a trapped electron with a trapped hole in the case of OSL (FIG. 2(b)). In both cases, the rate of change in the number or concentration of luminescence centers in the excited state as a function of time, m(t), can be written as:

$$\frac{d m ( t )}{dt} = pn ( t ) - {τ}^{- 1} m ( t ) , \qquad ( 3 )$$

The term pn(t) is the rate of excitation, n(t) representing either the number of centers in the ground state or the number of trapped charges, and the term τ⁻¹m(t) is the rate of de-excitation of the luminescence centers. The luminescence intensity is proportional to the rate of decay of the luminescence centers from the excited to the ground state:

I(t)∝τ⁻¹m(t), (4)

which is therefore directly proportional to m(t).

Continuing with the present embodiment and making the assumption that the number of fluorophores in the ground state or trapped charges does not change much during stimulation, i.e., that n≅n₀ during the short stimulation time, Eq. (3) can be integrated to obtain the following expression for the luminescence intensity during stimulation:

I(t)∝τ⁻¹m(t)=pn₀(1−e^{-1/τ}) (5)

After a stimulation period Δt, the luminescence decays exponentially with:

I(t)∝m(t)=pn₀(1−e^{-t/τ})e^{-(t-Δt)/τ}. (6)

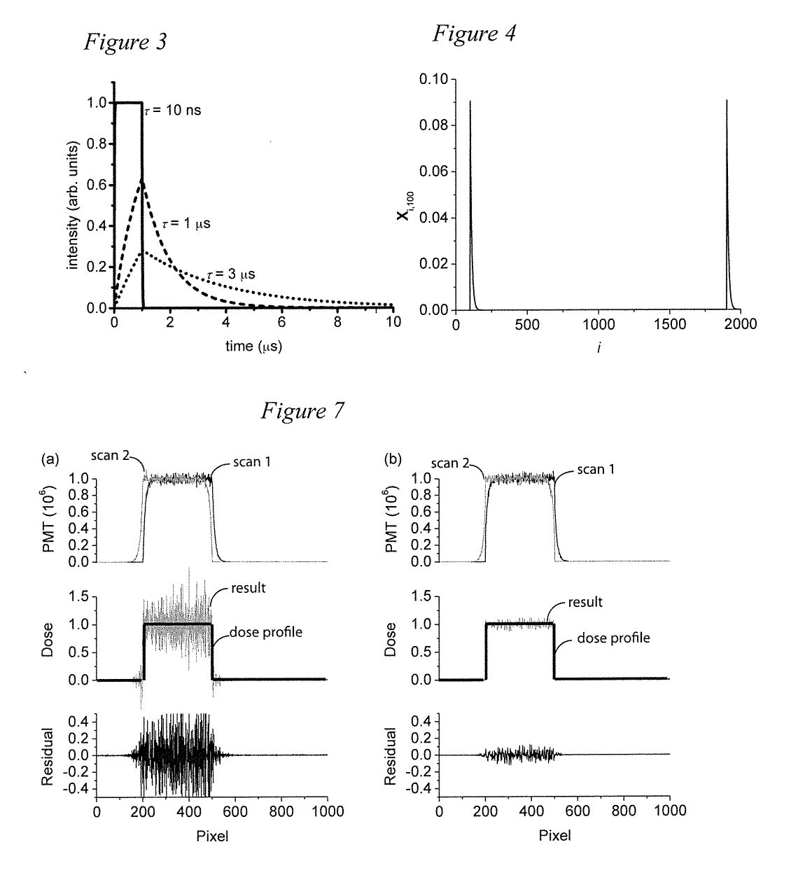

Equations (5) and (6) are plotted in FIG. 3 for three different values of the luminescence lifetime and a stimulation period of 1 μs. For luminescence centers with short lifetime, the luminescence is essentially concomitant with the stimulation. For luminescence lifetimes longer than the stimulation time, the luminescence persists for longer than the stimulation period.

### Deconvolution Procedure

To recover the dose profile based on the luminescence signal in the case of luminescence centers with lifetime τ, a least squares fit can be used to simultaneously estimate the signal obtained in one or in two opposite scan directions, similar to those shown in FIG. 1. In the case of phosphorescence, the scan can be in the same row. In the case of OSL, a second scan of the same row would result in lower intensity due to partial emptying of the trapped charges associated with the OSL signal. Although this could be modeled, here we assume the OSL scans in opposite directions to be taken in adjacent rows, so that the OSL intensity is not decreased but the dose profiles are approximately the same.

Define the response function g(t), which is the signal as a function of time when the laser scans a pixel with unitary dose, over the time interval from t=0 until t=Δt. Considering only a slow luminescence center, the response function has the same form as Eq. (5). For t>Δt, the signal decays exponentially as given by Eq. (6). Therefore, the shape of this response function is identical to the curves presented in FIG. 3.

Note that the choice of g(t) is appropriate when the material under stimulation is Al₂O₃:C or a compound with a similar response function. Those of ordinary skill in the art will understand how the techniques presented herein can readily be modified when the response function takes a different form from that utilized in the present example.

The discrete response gᵢ is then obtained by integrating g(t) between the intervals t=(i−1) Δt and i Δt. The result is:

$${g}_{i} = \{ \begin{matrix}0 & i < 1 \\ Δ t - τ ( 1 - {e}^{- Δ t / τ} ) & i = 1 \\ τ {{e}^{- \frac{i Δ t}{τ}} ( {e}^{- Δ t / τ} - 1 )}^{2} & i > 1\end{matrix} \qquad ( 7 )$$

If there are two luminescence centers contributing to the signal, one with lifetime much smaller than Δt that does not lead to bleeding, and one with lifetime τ and leads to bleeding, then Eq. (7) becomes:

$${g}_{i} = \{ \begin{matrix}0 & i < 1 \\ R + ( 1 - R ) [ Δ t - τ ( 1 - {e}^{- Δ t / τ} ) ] & i = 1 \\ ( 1 - R ) τ {{e}^{- \frac{i Δ t}{τ}} ( {e}^{Δ t / τ} - 1 )}^{2} & i > 1\end{matrix} , \qquad ( 8 )$$

where R is fraction of the total signal corresponding to the fast luminescence center and (1−R) is the fraction corresponding to the slow luminescence center. A similar modification to the equations can be made to account for more than two components with different luminescence lifetimes.

According to this embodiment, the signal will be written as:

y=Xa, (9)

where y is a column vector containing the final signal profile, X is the design matrix where each column j is the signal corresponding to a unit dose in pixel j and zero elsewhere, and a is column vector containing the information on the dose in each pixel, i.e., the “dose profile” or “image” that is to be recovered. Of course, those of ordinary skill in the art will recognize that the foregoing equation is of the sort generally associated with least squares linear regression. Thus, when the term “deconvolution” is used herein, that term should be broadly construed to include traditional deconvolution as well as any approach that can be characterized as a linear regression (least squares or any other norm-based regression or deconvolution) of the response function against the observed signal profile against the dose in each pixel.

When combining two scans, in this embodiment the combined scan y is simply the joined vectors yₐ and y_{b} containing the PMT signal for each scan direction. In this case, each column j in the design matrix X is the signal that would be expected for both scan directions for a unit dose at pixel i.

In terms of the response function gᵢ, the design matrix is given by:

$$\begin{matrix}{X}_{i , j} = \{ \begin{matrix}0 & i < j \\ {g}_{i - 1 + 1} & i \geq j\end{matrix} , for 1 \leq i \leq {n}_{p} \\ and & ( 10 ) \\ {X}_{i , j} = \{ \begin{matrix}0 & i + j - 2 {n}_{p} < 1 \\ {g}_{i - 1 - 2 {n}_{p}} & i + j - 2 {n}_{p} \geq 1\end{matrix} for {n}_{p} < i \leq 2 {n}_{p} & ( 11 )\end{matrix}$$

where nₚ is the number of pixels (data points) in each scan. Eq. (10) is the unit response to one scan direction and Eq. (11) is the unit response to the opposite scan direction. As an example, FIG. 4 plots the value of X for the column j=100. The values from i=1 to 1000 correspond to the scan in one direction for a unit dose in pixel j=100, whereas the data points from i=1001 to 2000 correspond to the scan in the opposite scan direction.

Therefore, Eq. (9) expresses the signal (column vector y) as the sum of the responses to a unit dose in each pixel j (columns in the design matrix X), multiplied by the dose at pixel j (column vector a). In principle, the column vector a can be found by (Gibbs, 2011):

a=(X^{T}X)⁻¹X^{T}y. (12)

If the data is considered to have uncertainties σ, then the solution can be written as:

a=(X^{T}V⁻¹X)⁻¹X^{T}V⁻¹y (13)

where V is the diagonal square matrix with diagonal elements corresponding to the variances σ² in the data points y. Here, the least squares problem was solved using the built-in function LeastSquares in Mathematica® 9.0.1.0 (Wolfram Research, Inc.).

Note that the term “inverting” will be used to describe obtaining corrected luminescence values by deconvolving the observed OSL values using a design matrix and, as specific examples, using equations of the form set out in equations (10) and (11) above to form the design matrix.

Needless to say, this scan could be repeated many different times along different scan lines if it were desired to analyze the entirety of the surface of a sample.

### Simulation Results

To test the procedure outlined above, it was applied to simulated data with various parameters and the results were compared.

For the simulations and according to one embodiment a one-dimensional detector was considered. It was divided into 1000 pixels and scanned with a dwell time Δt in two opposite directions (equivalent to the scan of adjacent rows characterized by the same dose profile in a 2D OSL detector). The dose profile was considered to be zero everywhere except between pixels 200 and 500, where the dose was constant equal to 1. A signal was then simulated that would be obtained in different conditions, assuming a background (0.1 counts pixel⁻¹), system sensitivity (a) in the 10²-10⁶ counts pixel⁻¹ unit dose⁻¹ range, and lifetimes in the (1-100) Δt range. The signal profiles were generated assuming a response function given by Eq. (8), but random noise following Poisson statistics was added to the data. The values used in the simulation were intended to simulate actual measurements using photomultiplier tube (PMT) counting. The parameter combinations used are shown in columns 2-4 in Table 1 below.

The simulated data was then processed using the least-squares algorithm described above to obtain the recovered dose distribution a. This was done assuming either equal weights to all data points (σ²=1; Eq. (12)), or a weighted least squares using the counts as the variance (σ²=y; Eq. (13)). A simple 2-point moving average was applied to remove some of the oscillation in the results. To compare the recovered dose distribution a to the original one, let i be the average absolute value of the residuals:

$$η = \frac{1}{{n}_{p}} {\sum }_{j} \vert {a}_{j} - {a}_{j , 0} \vert \qquad ( 14 )$$

where nₚ is the number of points (nₚ=1000), aⱼ is the recovered dose in the j-th pixel, and a_{j,0} is the actual dose in the j-th pixel. The maximum residuals observed (Δyₘₐₓ) were also calculated. The values of η and Δyₘₐₓ for each simulation are presented in Table 1.

TABLE 1
Simulation parameters and comparison of parameters η and Δyₘₐₓ
(average and maximum residuals, respectively) obtained using an
embodiment of the deconvolution algorithm presented above without
weighting (σ² = 1) and with weighting (σ² = y)
used in the least square minimization.

| | | α | | | | | |
|---|---|---|---|---|---|---|---|
| | | (counts | | | | | |
| | | pixel⁻¹ | | | | | |
| | | unit | τ | η | Δyₘₐₓ | η | Δyₘₐₓ |
| Set | R | dose⁻¹) | (Δt) | (σ² = 1) | (σ² = 1) | (σ² = y) | (σ² = y) |
| A-1 | 0 | 10⁶ | 1 | 2.6 × 10⁻⁴ | 0.003 | 2.6 × 10⁻⁴ | 0.003 |
| A-2 | 0 | 10⁶ | 10 | 2.5 × 10⁻³ | 0.028 | 2.2 × 10⁻³ | 0.025 |
| A-3 | 0 | 10⁶ | 100 | 3.5 × 10⁻² | 0.25 | 1.6 × 10⁻² | 0.22 |
| B-1 | 0 | 10³ | 1 | 8.0 × 10⁻³ | 0.14 | 8.6 × 10⁻² | 0.099 |
| B-2 | 0 | 10³ | 10 | 8.0 × 10⁻² | 1.0 | 8.0 × 10⁻² | 1.0 |
| B-3 | 0 | 10³ | 100 | 1.2 | 8.5 | 0.57 | 9.0 |
| C-1 | 0.3 | 10³ | 1 | 6.4 × 10⁻³ | 0.079 | 6.1 × 10⁻³ | 0.087 |
| C-2 | 0.3 | 10³ | 10 | 1.2 × 10⁻² | 0.12 | 1.0 × 10⁻² | 0.15 |
| C-3 | 0.3 | 10³ | 100 | 1.8 × 10⁻² | 0.14 | 1.0 × 10⁻² | 0.13 |
| D-1 | 0.7 | 10³ | 1 | 4.5 × 10⁻³ | 0.055 | 4.6 × 10⁻³ | 0.061 |
| D-2 | 0.7 | 10³ | 10 | 5.8 × 10⁻³ | 0.068 | 5.3 × 10⁻³ | 0.062 |
| D-3 | 0.7 | 10³ | 100 | 7.2 × 10⁻³ | 0.058 | 5.2 × 10⁻³ | 0.058 |
| E-1 | 0.3 | 10² | 1 | 2.0 × 10⁻² | 0.21 | 1.9 × 10⁻² | 0.25 |
| E-2 | 0.3 | 10² | 10 | 3.7 × 10⁻² | 0.45 | 3.7 × 10⁻² | 0.38 |
| E-3 | 0.3 | 10² | 100 | 6.0 × 10⁻² | 0.53 | 3.5 × 10⁻² | 0.38 |

### Influence of Luminescence Lifetime

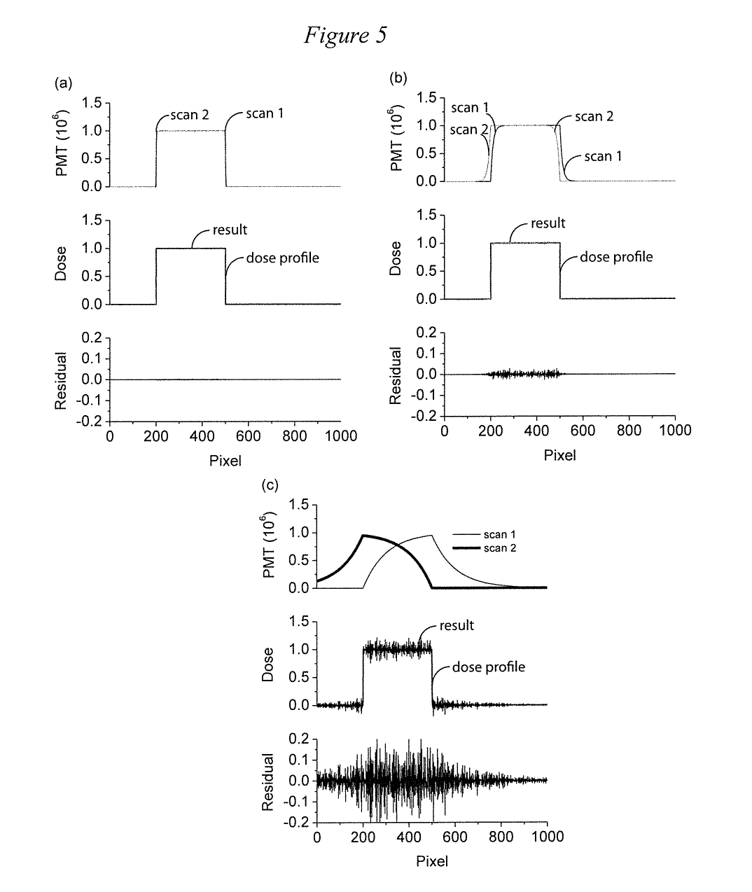

The influence of the luminescence lifetime is illustrated in the embodiment of FIG. 5, where the results are compared for three different lifetimes: (a) τ=Δt, (b) τ=10 Δt, and (c) τ=100 Δt (corresponding to simulations A-1, A-2, A-3). The top graphs show the simulated signal in both scan directions; the signal was assumed here to have minimum noise by considering 10⁶ counts per pixel in the non-zero dose region. As the lifetime increases from 1 Δt to 100 Δt, bleeding becomes more pronounced and the result from the deconvolution algorithm deteriorates. Nevertheless, in this “low noise scenario” the recovered dose profile agrees well with the dose profile even in the worst case of τ=100 Δt (FIG. 5(c)), the maximum residuals are of the order of 0.2, but could potentially be reduced by further data filtering.

If the system sensitivity is reduced from 10⁶ to 10³ counts pixel⁻¹ unit dose⁻¹, then the dose solutions oscillate too much, reaching maximum residuals Δyₘₐₓ>5 (see set B-3). The signal can still be partially recovered using smoothing techniques.

Mixture of Luminescence Centers with Slow and Fast Luminescence Lifetimes

If the detector contains a luminescence centers with fast luminescence lifetimes (τ<<Δt) in addition to slow luminescence centers, the results are improved.

FIG. 7 compares the results for the situation in which the detector has only slow luminescence centers with τ=10 Δt (FIG. 7(a)), or a combination of slow (τ=10 Δt) and fast luminescence centers (τ<<Δt) in a proportion R=0.3 (FIG. 7(b)), which means that a stimulated pixel will contribute with 30% through fast luminescence centers (not subjected to bleeding) and 70% through slow luminescence centers (subject to bleeding). One can see that even this small contribution from fast luminescence centers improves considerably the dose profile recovered. If the proportion of fast luminescence centers is increased to R=0.7, the results are improved even further, which can be seen comparing cases the results for the set C-3 and D-3 in Table 1 (reduction in Δyₘₐₓ by a factor of almost 3). It should be mentioned that the values of R=0.3 and R=0.7 were chosen to represent Al₂O₃:C and Al₂O₃:C,Mg, respectively.

### Weighting Data

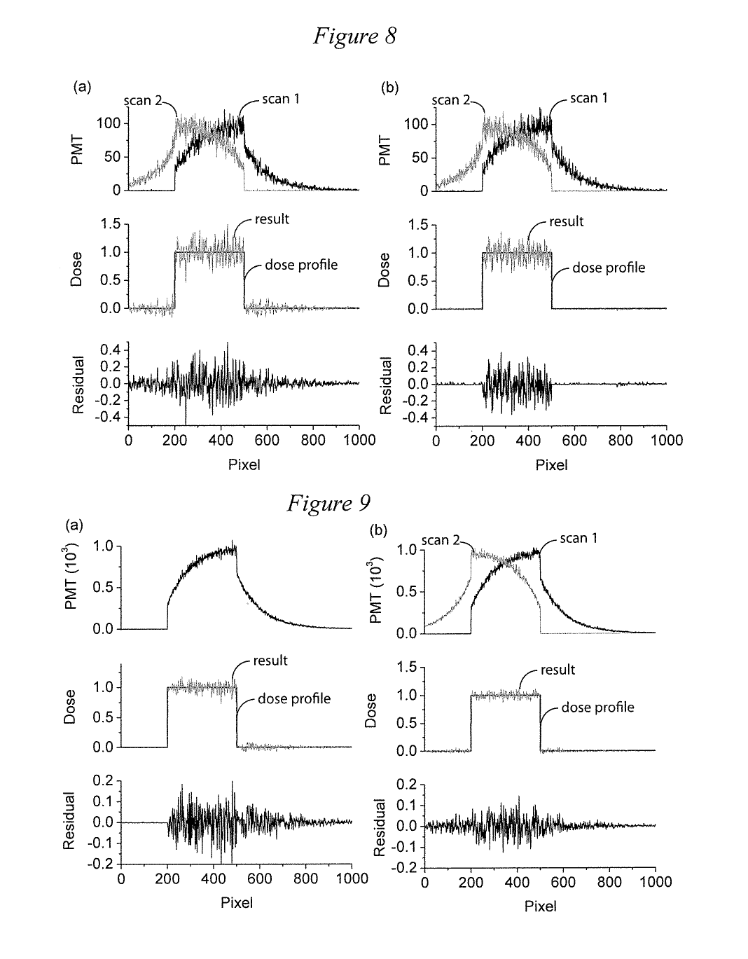

FIG. 8 compares the results for the simulation set E-3 (see Table 1) obtained using a least-squares method without weighting (σ²=1 for all data points; Eq. (12)) (FIG. 8(a)) or with weighting (σᵢ²=yᵢ for each data point; Eq. (13)) (FIG. 8(b)). Using the uncertainties in the data point as weight reduces the average amplitude of the residuals and decreases the maximum residuals observed (FIG. 8(b)). The results seem to be improved particularly in the zero dose region, but are not always considerable, as can be seen comparing the results with and without weighting in Table 1.

### Comparison Between Single Scan and Double Scan

FIG. 9 compares the results obtained if an embodiment of the algorithm is applied to a single scan (i.e., only one scan direction, FIG. 9(a)) or to a double scan (one scan in each direction, FIG. 9(b)). In this ideal case, the use of a single scan results in considerable noise after the laser had scanned a non-zero dose region. Use of the double scan creates a more symmetrical dose distribution around the non-zero dose region. The noise is also reduced because of the use of additional data points. Results for single scan simulations are presented in Table 2.

TABLE 2
Simulation parameters and comparison of parameters η
and Δyₘₐₓ (average and maximum deviations, respectively)
obtained using the deconvolution algorithm presented above without
weighting (σ² = 1) for the case of a single scan.

| Set | R | α | τ | η | Δyₘₐₓ |
|---|---|---|---|---|---|
| SC-1 | 0 | 10³ | 100 | 1.54 | 1100% |
| SC-2 | 0.3 | 10³ | 100 | 3.4 × 10⁻² | 30% |
| SC-3 | 0.7 | 10³ | 100 | 1.3 × 10⁻² | 12% |

### EXPERIMENTAL RESULTS

### 2D Scanning System

As an example of an embodiment, a flying-spot laser scanning system was constructed using a 2D Galvo mirror system (Model GVS002, Thorlabs, Inc.) and a 532 nm DPSS laser (Model: GMLN-532-100FED, output power 100 mW, Lasermate Group, Inc.) (Ahmed et al., 2014). The laser light is focused on the detector using a piano-convex lens (400 mm focal length, model KPX115, Newport Corporation). The OSL detectors were placed on top of a 15.2 cm×15.2 cm×3 mm long-pass glass filter (GG495, Schott Glass Corporation), through which the laser light was transmitted. On top of the OSL detectors was a band-pass filter (2.5 mm thickness Hoya U-340 or Hoya B-370, depending on the experiment, Hoya Corporation) to keep them flat. For detection of only the F-center OSL emission, the OSL was detected through a total of 7.5 mm Hoya B-370 filter+2.5 mm Schott BG-39 filter (Schott AG); for detection of a combination of F⁺ and F-center, the OSL was detected through a total of 7.5 mm Hoya U-340 filters.

A photomultipler tube (PMT) was used as the light transducer (51 mm diameter, model 9235QA, Electron Tubes, Inc.). The PMT was operated in photon counting mode using a multichannel scaler (SR-430, Stanford Research), which resolves counts in each row into 1024 bins with 327 μs bin width. The imaging area was scanned in successive rows separated by 0.167 mm in alternating directions. The laser power is monitored using a Si biased photodiode (model DET10A, Thorlabs, Inc.) using a beam splitter. For the dataset presented in this paper, the laser power variation was less than 0.2% (1SD).

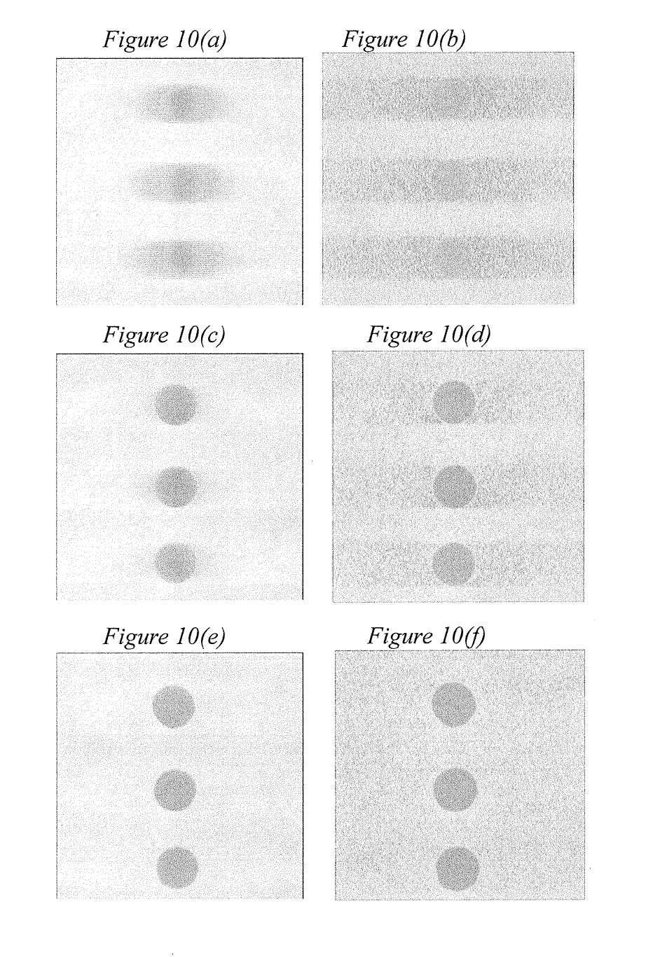

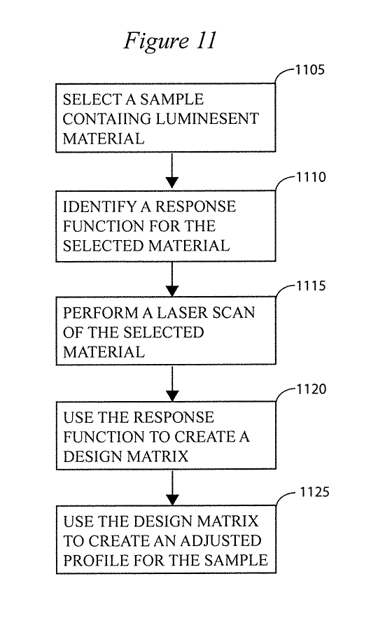

To avoid saturating the PMT, the images obtained using Hoya B-370 filter (FIG. 10(a) and FIG. 10(b)) were obtained by lowering the excitation power to 10% (OD 1.0 neutral density filter was placed in the laser path) of original power that was used for other images.

All the irradiations were performed using a 100 mCi ⁹⁰Sr/⁹⁰Y beta source delivering a dose rate of ˜1 mGy/s for 1000 s (˜1 Gy dose). Before irradiation, all the samples were bleached for 15 min using green LEDs to eliminate any native OSL signal.

### Samples

In this work Al₂O₃:C film identical to those used in the Luxel+ dosimetry system was used, but cut into discs 7 mm in diameter, and a proprietary Al₂O₃:C,Mg film produced with particle size <38 μm, also cut into discs 7 mm in diameter.

### Correction for Pixel Bleeding

Correction of the images for pixel bleeding was performed as in the case of the simulations, except that the algorithm is performed for several R values. The value of R that minimizes the residuals is then chosen for the pixel bleeding correction. The lifetime for each material was determined based on the luminescence decay part (tails) of the dose profiles.

### Results

FIG. 10 shows the images for different types of Al₂O₃ irradiated discs before (left side) and after correction (right side) using the algorithm proposed here. The first row of images (FIGS. 10(a) and 10(b)) shows the results for Al₂O₃:C measuring the F-center luminescence (Hoya B-370 filter); the second row (FIGS. 10(c) and 10(d)) show the results for Al₂O₃:C measuring a combination of F and F⁺ center (Hoya U-340 filter); the third row (FIGS. 10(e) and 10(f)) show the results for Al₂O₃:C,Mg also measuring a combination of F and F⁺ center (Hoya U-340 filter). The images were obtained scanning each row in alternating directions.

When only F-center emission is detected (FIGS. 10(a) and 10(b)), the pixel bleeding is very strong and the shape of the OSL discs are barely recognizable before correction (FIG. 10(a)). After correction (FIG. 10(b)), the shape of the OSL discs is clear, but there is considerable noise in the image. The advantage of this filter combination is that the signal intensity is very high. In this case the laser power had to be decreased to measure these images without saturating the PMT.

When a combination of F- and F⁺-center emission is detected (FIGS. 10(c) and 10(d)), the images are considerably improved even before correction, although pixel bleeding can still be observed (FIG. 10(c)). After correction (FIG. 10(d)), the image of the OSL discs is well defined. So, this filter combination improves the special resolution and reduces noise for these scan parameters, but the signal detected is weaker: the peak counts per pixel was ˜250. Nevertheless, this image demonstrates the possibility of correcting for the F-center emission even when the pixel dwell time is 300 times faster (327 μs) than what would be required considering the luminescence lifetime of the F-centers (35 ms), assuming the three lifetime requirement.

Finally, when a material with higher F⁺-center emission is used and a combination of F- and F⁺-center emission is detected (FIGS. 10(e) and 10(f)), the correction algorithm becomes less important, but is still required to correct for the F-center contribution.

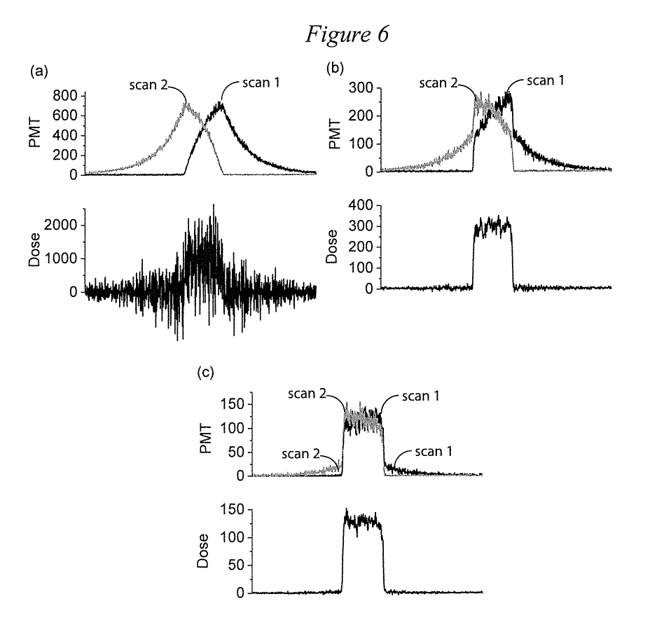

The performance of the algorithm and noise level can be better visualized in FIG. 6, which shows the scan of two rows along the middle part of one of the OSL discs for the three different cases: (a) Al₂O₃:C measuring F-center emission (Hoya B-370+Schott BG-39 filter), (b) Al₂O₃:C measuring a combination of F- and F⁺-center emission (Hoya U-340); and (c) Al₂O₃:C,Mg also measuring a combination of F- and F⁺-center emission (Hoya U-340). It is clear in FIG. 6 the different influence of F-center emission and, consequently, of pixel bleeding for the different filter and material combinations. The level of noise is also clear. In FIG. 6(a), the dose profile cannot be seen without further noise reduction; in FIG. 6(c) the tails in the PMT signal due to F-centers have been corrected using the filtering algorithm.

### DISCUSSION

There is taught herein a method of correcting for the influence of luminescence centers with slow luminescence lifetime in laser scanning imaging, with such embodiment being illustrated using both numerical simulations and experimental results for the case of the OSL signal from Al₂O₃. In addition, the performance of the an embodiment of the method in different conditions was investigated, e.g., in the presence of only a slow luminescence center, in the presence of a combination of slow and fast luminescence centers.

The results not only demonstrate the possibility of using such algorithm in imaging and 2D dosimetry applications, but it also highlight the versatility of using laser scanning for reading out the OSL from Al₂O₃:C. Laser scanning provides the following advantages: (a) the laser power can be varied to control the OSL stimulation rate (to allow for better control of signal loss for consecutive readouts or to prevent PMT saturation); (b) the laser scan rate can be varied according to the application, potentially enabling higher precision and dynamic range at slower rates versus lower precision and lower dynamic range at faster rates; (c) the spatial resolution can be controlled by the laser spot size and scan rate, and it is not determined only by the detector resolution (as in the case of CCD-based readers).

It was also showed that, even in situations when there is a dominant fast luminescence center, as in the case of Al₂O₃:C,Mg measured using Hoya U-340 filters, correction for the presence of slow luminescence centers is still necessary. An approach of the sort taught herein makes it possible.

### CONCLUSIONS

According to one embodiment, the instant disclosure teaches an algorithm for correcting for the presence of centers characterized by slow luminescence lifetime in laser scanning imaging applications. The viability of this embodiment was verified using numerical results and experimental data on the optically stimulated luminescence from Al₂O₃:C. An embodiment of the algorithm allows the imaging of OSL Al₂O₃ detectors 300 times faster than what would be required considering the lifetime of the main luminescence centers in Al₂O₃, which is ˜35 ms (assuming a required dwell time of three lifetimes). By relaxing the stringent requirements on the luminescence lifetime of the detector materials, this variation opens the possibility of using new materials in 2D dosimetry as well as other laser scanning applications, such as X-ray imaging using storage phosphors and scanning confocal microscopy.

Note that when the term “matrix” is used herein, that term should be broadly construed to include arrays of numbers that consist of a single column, arrays of numbers that are two dimensional (i.e., containing both rows and columns), as well as three dimensional (i.e., containing rows, columns, and a third dimension).

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

For purposes of the instant disclosure, the term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Each of the references identified herein is incorporated by reference into this document as if fully set out at this point.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

### REFERENCES

Ahmed, M. F., Eller, S., Schnell, E., Ahmad, S., Akselrod, M. S. and Yukihara, E. G., 2014. Development of a 2D dosimetry system based on the optically stimulated luminescence of Al₂O₃. Radiat. Meas. 71, 187-192.
Akselrod, G. M., Akselrod, M. S., Benton, E. R. and Yasuda, N., 2006. A novel Al₂O₃ fluorescent nuclear track detector for heavy charged particles and neutrons. Nucl. Instrum. Methods. Phys. Res. B 247, 295-306.
Akselrod, M. S., Lucas, A. C., Polf, J. C. and Mckeever, S. W. S., 1998. Optically stimulated luminescence of Al₂O₃. Radiat. Meas. 29, 391-399.
Beiser, L. and Johnson, R. B., 2010. Scanners. In: Handbook of Optics, M. Bass, V. N. Mahajan (Eds.). McGraw Hill, New York, 30.31-30.68.
Bøtter-Jensen, L., McKeever, S. W. S. and Wintle, A. G., 2003. Optically Stimulated Luminescence Dosimetry. Elsevier, Amsterdam.
Claxton, N. S., Fellers, T. J. and Davidson, M. W., 2006. Microscopy, Confocal. In: Encyclopedia of Medical Devices and Instrumentation. John Wiley & Sons, Inc.
Evans, B. D. and Stapelbroek, M., 1978. Optical properties of the F⁺ center in crystalline Al₂O₃. Phys. Rev. B 18, 7089-7098.
Gibbs, B. P., 2011. Advanced Kalman Filtering, Least-Squares and Modeling. John Wiley & Sons, Inc., Hoboken, N. J.
Han, Z. H., Driewer, J. P., Zheng, Y. S., Low, D. A. and Li, H. H., 2009. Quantitative megavoltage radiation therapy dosimetry using the storage phosphor KCl:Eu2+. Med. Phys. 36, 3748-3757.
Idri, K., Santoro, L., Charpiot, E., Herault, J., Costa, A., Ailleres, N., Delard, R., Vaille, J. R., Fesquet, J. and Dusseau, L., 2004. Quality control of intensity modulated radiation therapy with optically stimulated luminescent films. IEEE Trans. Nucl. Sci 51, 3638-3641.
Jahn, A., Sommer, M. and Henniger, J., 2010. 2D-OSL-dosimetry using beryllium oxide. Radiat. Meas. 45, 674-676.
Jahn, A., Sommer, M., Liebmann, M. and Henniger, J., 2011. Progress in 2D-OSL-dosimetry with beryllium oxide. Radiat. Meas. 46, 1908-1911.
Lee, K. H. and Crawford Jr., J. H., 1979. Luminescence of the F center in sapphire. Phys. Rev. B 19, 3217-3221.
Li, H. H., Xiao, Z. Y., Hansel, R., Knutson, N. and Yang, D. S., 2013. Performance of KCl:Eu2+ storage phosphor dosimeters for low-dose measurements. Phys. Med. Biol. 58, 4357-4366.
McKeever, S. W. S., 2001. Optically stimulated luminescence dosimetry. Nucl. Instrum. Methods. Phys. Res. B 184, 29-54.
Olch, A. J., 2005. Evaluation of a computed radiography system for megavoltage photon beam dosimetry. Med. Phys. 32, 2987-2999.
Rodriguez, M. G., Denis, G., Akselrod., M. S., Underwood, T. H. and Yukihara, E. G., 2011. Thermoluminescence, optically stimulated luminescence and radioluminescence properties of Al₂O₃:C,Mg. Radiat. Meas. 46, 1469-1473.
Rowlands, J. A., 2002. The physics of computed radiography. Phys. Med. Biol. 47, R123-R166.
Sheppard, C. and Shotton, D., 1997. Confocal Laser Scanning Microscopy. Springer, New York.
Tsien, R. Y., Ernst, L. and Waggoner, A., 2006. Fluorophores for Confocal Microscopy: Photophysics and Photochemistry. In: Handbook of Biological Confocal Microscopy, J. B. Pawley (Ed.). Springer Science+Business Media, New York.
von Seggern, H., 1999. Photostimulable X-ray storage phosphors: a review of present understanding. Braz. J. Phys 29, 254-268.
Yukihara, E. G. and McKeever, S. W. S., 2006. Spectroscopy and optically stimulated luminescence of Al2O3:C using time-resolved measurements. J. Appl. Phys. 100, 083512.
Yukihara, E. G. and McKeever, S. W. S., 2008. Optically Stimulated Luminescence (OSL) dosimetry in Medicine. Phys. Med. Biol. 53, R351-R379.
Yukihara, E. G. and McKeever, S. W. S., 2011. Optically Stimulated Luminescence: Fundamentals and Applications. John Wiley & Sons, Chichester, West Sussex, UK.

What is claimed is:

1. A method of laser scanning imaging a sample containing a luminescent material, comprising the steps of:
(a) selecting a response function for said luminescent material;
(b) performing a laser scan of said sample, thereby obtaining a plurality of luminescence values;
(c) using said response function of said luminescent material to form a design matrix corresponding to said laser scan; and,
(d) using said design matrix and said plurality of luminescence values to calculate an adjusted profile for said sample.

2. A method of laser scanning imaging a sample containing a luminescent material according to claim 1, wherein step (d) comprises the step of calculating
a=(X^{T}X)⁻¹X^{T}y,
where,
a is a column vector containing said adjusted profile for the sample,
y is a column vector containing said plurality of luminescence values from said laser scan of said sample, and,
X is said design matrix.

3. A method of laser scanning imaging a sample containing a luminescent material according to claim 1, wherein step (d) comprises the step of calculating
a=(X^{T}V⁻¹X)⁻¹X^{T}V⁻¹y,
where,
a is a column vector containing said adjusted profile for the sample,
y is a column vector containing said plurality of luminescence values from said laser scan of said sample,
X is said design matrix,
σ² is a variance of said plurality of luminescence values from said laser scan of said sample,
V is a diagonal square matrix with diagonal elements comprising said σ².

4. A method of laser scanning imaging a sample containing a luminescent material, according to claim 1, wherein step (d) comprises the step of calculating an adjusted profile for said sample by deconvolution using said design matrix and said plurality of luminescence values.

5. A method of performing 2D dosimetry on a sample of a luminescent material, comprising the steps of:
(a) selecting a response function for said luminescent material;
(b) obtaining a plurality of laser scans of the sample, thereby obtaining a plurality of luminescence values;
(c) using said response function of said luminescent material to form a design matrix corresponding to each of said plurality of laser scans; and,
(d) using said design matrix and said plurality of luminescence values to calculate an adjusted OSL dose profile for said sample using deconvolution.

6. A method of performing 2D dosimetry on a sample of a luminescent material according to claim 5, wherein step (d) comprises the step of calculating
a=(X^{T}X)⁻¹X^{T}y,
where,
a is a column vector containing said adjusted OSL dose profile for the sample,
y is a column vector containing said plurality of OSL values from said plurality of laser scans of said sample, and,
X is said design matrix.

7. A method of performing 2D dosimetry on a sample of a luminescent material according to claim 5, wherein step (d) comprises the step of calculating
a=(X^{T}V⁻¹X)⁻¹X^{T}V⁻¹y,
where,
a is a column vector containing said adjusted OSL dose profile for the sample,
y is a column vector containing said plurality of OSL values from said laser scan of said sample,
X is said design matrix,
σ² is a variance of said plurality of OSL values from said laser scan of said sample,
V is a diagonal square matrix with diagonal elements comprising said σ².

8. A method of performing 2D dosimetry on a sample of a luminescent material according to claim 5, wherein step (d) comprises the step of calculating an adjusted profile for said sample by deconvolution using said design matrix and said plurality of luminescence values.

9. A method of laser scanning imaging a sample containing a luminescent material,
comprising the steps of:
(a) selecting a response function for said luminescent material;
(b) performing a first laser scan of said sample in a first direction, thereby obtaining a first plurality of luminescence values;
(c) performing a second laser scan of said sample in a direction opposite said first direction, thereby obtaining a second plurality of luminescence values;
(d) using said response function of said luminescent material to form a design matrix corresponding to said first laser scan direction and said second laser scan direction; and,
(e) using said design matrix and said first and second plurality of luminescence values to calculate an adjusted profile for said sample.

10. A method of laser scanning imaging a sample containing a luminescent material according to claim 9, wherein step (e) comprises the step of calculating
a=(X^{T}X)⁻¹X^{T}y,
where,
a is a column vector containing said adjusted profile for the sample,
y is a column vector containing said first and second plurality of luminescence values from said first and second laser scans of said sample, and,
X is said design matrix.

11. A method of laser scanning imaging a sample containing a luminescent material according to claim 9, wherein step (e) comprises the step of calculating
a=(X^{T}V⁻¹X)⁻¹X^{T}V⁻¹y,
where,
a is a column vector containing said adjusted profile for the sample,
y is a column vector containing said first and second plurality of luminescence values from said first and second laser scan of said sample,
X is said design matrix,
σ² is a variance of said first and second plurality of luminescence values from said first and second laser scans of said sample,
V is a diagonal square matrix with diagonal elements comprising said σ².

12. A method of laser scanning imaging a sample containing a luminescent material according to claim 9, wherein step (e) comprises the step of calculating an adjusted profile for said sample by deconvolution using said design matrix and said first and second plurality of luminescence values.
